(19) United States
(12) Patent Application Publication
(10) Pub. No.: US 2005/0281858 A1
(43) Pub. Date: Dec. 22, 2005

(54) Devices, articles, coatings, and methods for controlled active agent release

(76) Inventors: Tim M. Kloke, Chaska, MN (US); Ralph A. Chappa, Prior Lake, MN (US)

Correspondence Address: MERCHANT & GOULD PC, P.O. BOX 2903, MINNEAPOLIS, MN, 55402-0903, US

(21) Appl. No.: 11/156,245

(22) Filed: Jun. 17, 2005

Related U.S. Application Data
(60) Provisional application No. 60/580,918, filed on Jun. 18, 2004.

(51) Int. Cl. A61K 9/46

(52) U.S. Cl. 424/423

(57) ABSTRACT

The present invention relates to multi-layer coatings and device, articles, and methods regarding the same, for controlled active agent release. Embodiments of the present invention include devices, articles, coatings, and methods relating to an composition including an active agent, a first layer disposed on the composition, and a second layer disposed on the first layer, wherein the second layer is configured to provide controlled release of the active agent through the second layer and the second layer has release characteristics that are distinct from the first layer.

This application claims the benefit of U.S. Provisional Application No. 60/580,918, filed Jun. 18, 2004, the contents of which is herein incorporated by reference.

### FIELD OF THE INVENTION

The present invention relates to devices, articles, coatings, and methods for controlled active agent release. More specifically, the present invention relates to multi-layer coatings and devices, articles, and methods regarding the same for controlled active agent release.

### BACKGROUND OF THE INVENTION

Therapeutic benefits can be achieved in some instances by providing an active agent to a subject in a manner that extends the time over which the active agent is released. Further, therapeutic benefits can be achieved by providing an active agent to a specific target tissue, instead of systemically. This is because the effect of the agent on the target tissue can be maximized while limiting side effects on other tissues. One approach to providing these benefits is to use a coating system containing an active agent on a medical device. The coating can serve to control the rate at which an active agent is eluted while the fact that it is on a medical device allows the delivery to be in proximity to specific tissues.

However, current coating systems do not perform well with some types of active agents. Some active agents may elute through current coating systems too quickly, others may not elute fast enough. This is partly because active agents are very diverse in their chemical properties including size, hydrophobicity, charge, etc, and these properties can affect their interaction with the coating system components. For example, small hydrophilic drugs such as Trigonelline HCL, diclofenac, and chlorhexidine diacetate typically elute with large initial bursts from current coating systems and therefore demonstrate poor elution rate control.

Further, some coating systems allow active agents to migrate through the coating layers to aggregate in regions of higher active agent concentration. Crystals of the active agents may form in these regions of higher concentration. Crystals may form when a coating system is formed or when a coating system is exposed to an aqueous environment. However, crystals may be undesirable because they can prevent effective control of the elution profile. This is because crystal formation can promote a larger variance among drug-coated devices that are similarly manufactured.

Some coating systems employ the use of vapor or plasma deposited self-initiating polymers. However, the deposition of such polymers over an active agent can allow the interaction of free radicals with the active agent creating potentially undesirable by-products.

Coating systems are frequently employed on medical devices configured for insertion into a subject. By way of example, a coating system may be used over a stent. Many medical devices must be flexible as they are deformed in the course of use. Therefore, the coating system used on such a device should be able to maintain its integrity during and after the deformation of the device. However, some current coating systems have difficulty adhering to a medical device that is deformed in the course of use.

Therefore, a need exists for a coating system that will work with many active agents. A need exists for a coating system that will not expose an active agent to free radicals during fabrication. Also, a need exists for a coating system that will adhere to a substrate properly.

### SUMMARY OF THE INVENTION

The present invention relates to devices, articles, coatings, and methods for providing controlled active agent release. Embodiments of the present invention include devices, articles, coatings, and methods including a composition including an active agent, a first layer disposed on the composition, and a second layer disposed on the first layer. The second layer can be configured to provide controlled release of the active agent from the device or article. The second layer can have release characteristics that are distinct from the first layer. Embodiments of the present invention also include devices, articles, coatings, and methods including a composition including an active agent, a first polymeric layer disposed on the composition, and a second polymeric layer disposed on the first polymeric layer. The first polymeric layer can include or be a pre-polymerized solvent-deposited polymer. The second polymeric layer can include or be a self-initiating polymer.

The above summary of the present invention is not intended to describe each discussed embodiment of the present invention. This is the purpose of the figures and the detailed description that follows.

### DRAWINGS

The invention may be more completely understood in connection with the following drawings, in which:

FIG. 1 an exemplary coated medical device in accordance with an embodiment of the invention.

FIG. 2 is a cross-sectional view of the coated medical device of FIG. 1 taken along line A-A′.

FIG. 3 is an enlarged cross-sectional view of elements of FIG. 2.

FIG. 4a is a cross-sectional view of a layered coating in accordance with an embodiment of the invention.

FIG. 4b is a cross-sectional view of a layered coating in accordance with another embodiment of the invention.

FIG. 5 is a cross-sectional view of an article in accordance with an embodiment of the invention.

FIG. 6 is a graph showing the elution profiles of a hydrophilic agent from various coatings in accordance with an embodiment of the invention.

FIG. 7 is a graph showing the elution profiles of a hydrophobic agent from various coatings in accordance with another embodiment of the invention.

FIG. 8 is a graph showing the elution profiles of a hydrophilic agent from various coatings in accordance with an embodiment of the invention.

FIG. 9a shows the repeating subunit of parylene-C.

FIG. 9b shows the repeating subunit of parylene-N.

FIG. 9c shows the repeating subunit of parylene-D.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Embodiments of the present invention can provide controlled release of active agents. Embodiments of the present invention relate to devices, articles, coatings, and methods that can provide controlled active agent release.

It has been surprisingly discovered that a coat of a vapor or plasma deposited polymer separated from an active agent by a separating layer can result in the active agent having a desirable elution profile. By way of example, hydrophilic active agents can be released with a desirable profile. Moreover, because the active agent can be covered by the separating layer during the process of applying the vapor or plasma deposited polymer, the exposure of the active agent to free radicals that could react with the active agent and form potentially undesirable contaminants can be reduced. The separating layer can also help to promote adhesion between the agent and the coat of a vapor or plasma deposited polymer.

The active agent can be accompanied by a carrier component. The active agent can be provided in the form of an active agent layer. However, the active agent can also be provided in a form that is not a layer. The active agent, or active agent layer, may or may not be deposited on a substrate.

As used herein, the term “separate” means to set or keep apart. In some instances, with respect to at least two objects, separate means to prevent from being immediately adjacent, and thereby come between the at least two objects. In this manner, the separating entity can cause adherence between the at least two objects. As the objects are kept apart, the term separate can, in certain embodiments, include that one object is protected from the other object.

The term separating layer refers to a layer that keeps apart at least two objects at least in one place. Accordingly, the term separating layer includes a layer that keeps apart two objects in a particular place but not in all places. The term continuous separating layer refers to a layer that keeps apart at least two objects in all places except for incidental spots that may occur, for example, spots that occur because of manufacturing defects.

The separating layer may also be referred to as a first layer. In embodiments where the separating layer comprises a polymeric material, such as a preformed polymer, the separating layer may also be referred to as a first polymeric layer. In embodiments where the separating layer provides protection the separating layer may be referred to as a protective layer.

The elution control layer may be referred to as a second layer. In embodiments where the material disposed on the separating layer comprises a polymeric material, such as a vapor or plasma deposited polymer, the material may be referred to as a second polymeric layer.

In an embodiment, the present invention includes a medical device including a structure configured for introduction into a subject, a composition including an active agent disposed on the structure, a first layer disposed on the composition, and a second layer disposed on the first layer. The second layer can be configured to provide controlled release of the active agent. The second layer can have release characteristics that are distinct from the first layer. In an embodiment, the invention relates to a medical device including a structure configured for introduction into a subject, a composition including an active agent disposed on the structure, a first polymeric layer disposed on the agent layer, and a second polymeric layer disposed on the first polymeric layer. The first polymeric layer can include a pre-polymerized solvent deposited polymer. The second polymeric layer can include a self-initiating polymer.

In an embodiment, the invention relates to a coating including a base layer with an active agent, a first polymeric layer disposed on the base layer, and a second polymeric layer disposed on the first polymeric layer. The first polymeric layer can be configured to separate the active agent from the second polymeric layer. The second polymeric layer can be configured to provide controlled release of the active agent and can have release characteristics that are distinct from the first polymeric layer. In an embodiment, the invention can be a coating including a base layer containing a bioactive material, a first polymeric layer disposed on the base layer, and a second polymeric layer disposed on the first polymeric layer. The first polymeric layer can include a pre-polymerized solvent deposited polymer. The second polymeric layer can include a self-initiating polymer.

In an embodiment, the invention can be an article including a core containing an active agent, a first polymeric layer disposed on the core, and a second polymeric layer disposed on the first polymeric layer. The first polymeric layer can be configured to separate the active agent from the second polymeric layer. The second polymeric layer can be configured to provide controlled release of the active agent and have release characteristics that are distinct from the first polymeric layer. In an embodiment, the invention can be an article including a core containing a bioactive material, a first polymeric layer disposed on the core, and a second polymeric layer disposed on the first polymeric layer. The first polymeric layer can include a pre-polymerized solvent deposited polymer. The second polymeric layer can include a self-initiating polymer.

In an embodiment, the invention can be a method for producing an article that provides controlled release of a hydrophilic active agent. This method can include depositing a base including a hydrophilic active agent, depositing a separating layer on the base, and depositing an elution control layer on the protective layer. The elution control layer can have release characteristics that are distinct from the protective layer.

In an embodiment, the present composition can provide prolonged release of an active agent. The present composition can be used to elute an active agent in a linear manner. The present composition can also be used to elute an active agent so as to have a reverse-burst elution profile. As used herein, the term “reverse-burst elution profile” means an elution profile which does not have a typical burst profile. The term reverse-burst elution profile can encompass an elution profile characterized by a lag where an insignificant amount of active agent elutes followed by a period where elution increases as a curve. For example, the term reverse-burst elution profile can include the profile where elution begins slowly relative to a later accelerated elution rate. By way of example, a reverse burst elution profile may exist where more of a given compound is eluted during days 11-20 than during days 1-10. A reverse burst elution profile may also exist where more of a given compound is eluted during days 6-10 than during days 1-5. The term “extended release”, as used herein, refers to an elution profile wherein the release of an active agent is prolonged as compared with a similar coating lacking an elution control layer. Embodiments of the invention include those having extended release profiles. The term “reduced burst”, as used herein, refers to an elution profile where the initial release burst is significantly reduced as compared with a similar coating lacking an elution control layer. Embodiments of the invention include those having reduced burst release profiles.

Further, it is believed that a coat of a vapor or plasma deposited polymer can reduce or prevent migration of an active agent to the surface of a coating and reduce or prevent formation of crystals on the surface of a coating.

U.S. Pat. No. 5,563,056 (Swan et al.), U.S. Pat. No. 6,214,901 (Chudzik et al.), U.S. published application Number 20020041899 (Chudzik et al.), U.S. published application Number 20020188037 (Chudzik et al.), and U.S. published application Number 20030129130 (Guire et al.), are all herein incorporated by reference.

Referring to FIG. 1, a view of an exemplary coated medical device 10 in accordance with an embodiment of the invention is shown. In this case, the medical device is a stent. However, one of skill in the art will appreciate that many different kinds of devices can be coated in accordance with embodiments of the invention. Other exemplary devices are described below. The medical device has a first end 12 and a second end 14. In between the first end 12 and the second end 14 the device includes a plurality of stainless steel wires 16 (or threads). In an embodiment, the metal wires are coated with an agent, an intermediate layer, and an outer layer (not shown).

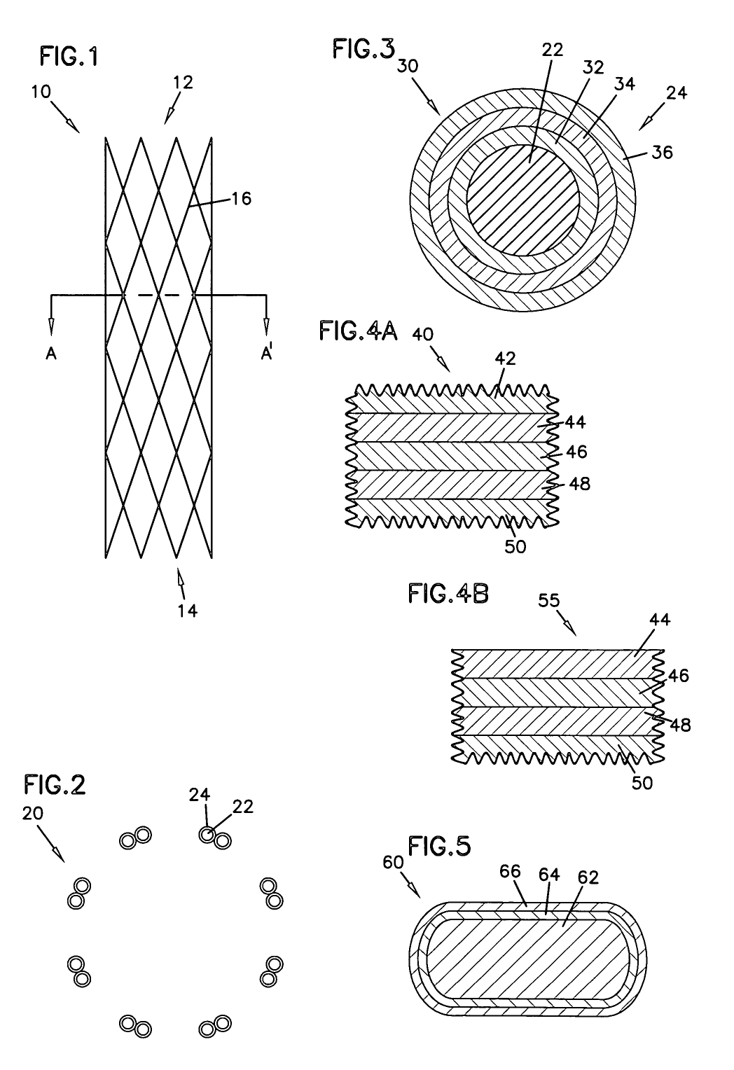

Referring now to FIG. 2, a cross-sectional view 20 of the coated medical device of FIG. 1 taken along line A-A′ is shown. In this view, the substrate 22 of the medical device 20 is shown, which in this case is stainless steel. A multi-layer coating 34 is disposed on the substrate 22.

FIG. 3 is an enlarged cross-sectional view 30 of elements of FIG. 2, wherein the multiple layers of the multi-layer coating 24 can be seen. In the middle of the cross-section is the substrate 22. A composition 32 including an active agent, or an agent layer including an active agent and a carrier component is disposed on the substrate 22. In an embodiment, the carrier component can be any material that provides adhesion to the substrate 22. The composition 32 can be just an active agent by itself. The composition 32 may also comprise an excipient. In an embodiment, the active agent or agent layer is deposited on the substrate 22 via a solvent deposition process. However, many different deposition processes can be used. In an embodiment, the carrier component includes a carrier polymer. The carrier polymer can be a preformed polymer as described below.

An intermediate layer 34, is disposed on the agent 32 or the agent layer. The intermediate layer 34 can be any material which provides adhesion and protection. In certain embodiments, the intermediate layer can include a preformed polymer as described below. In an embodiment, the intermediate layer can include poly(n-butyl methacrylate) and poly(ethylene-co-vinyl acetate).

An outer layer 36, is disposed on the intermediate layer 34. The outer layer 36 can include a self-initiating polymer. In an embodiment, the outer layer 36 can include a vapor or plasma deposited layer. In an embodiment, the outer layer 36 can include at least one of poly 2-chloro-paraxylylene (parylene C), polyparaxylylene (parylene N), and poly 2,5-dichloro-paraxylylene (parylene D). In a particular embodiment, the outer layer 36 can include poly 2-chloro-paraxylylene (parylene C).

Referring now to FIG. 4a, a cross-sectional view of a layered coating 40 in accordance with an embodiment of the invention is shown. An agent 48, or an agent layer, including an active agent can be disposed on an underlying element 50. In an embodiment, the underlying element 50 can include a variety of articles or surfaces, for example, a second agent layer, an adhesive layer, a spacing layer, a polymeric layer, a substrate, or more than one of these. An intermediate layer 46, is disposed on the agent 48 or the agent layer. An outer layer 44 is disposed on the intermediate layer 46. An overlying element 42, can be disposed on the outer layer 44. In an embodiment, the overlying element 42 can include a second agent layer, an adhesive layer, a spacing layer, a polymeric layer, a substrate, or more than one of these. Referring now to FIG. 4b, a cross-sectional view of a layered coating in accordance with another embodiment of the invention is shown. The layered coating 55 is the same as the layered coating 40 of FIG. 4a except that this embodiment does not have an overlying element.

Referring now to FIG. 5, a cross-sectional view of an article 60 in accordance with an embodiment of the invention is shown. A core 62, or base, including an active agent is at the interior of the article. An intermediate layer 64 is disposed on the core 62. An outer layer 66 is disposed on the intermediate layer 64.

Components of embodiments of the invention will now be described in greater detail.

Polymers of the Agent Layer And Separating Layer

In some embodiments of the invention, the agent layer (or base layer) can include a preformed polymer. For example, an active agent may be mixed with a preformed polymer and then deposited on a substrate.

In some embodiments, the separating layer can include a preformed polymer. As discussed above, the separating layer may also be referred to as a first layer. In embodiments where the separating layer comprises a polymeric material, such as a preformed polymer, the separating layer may also be referred to as a first polymeric layer. In embodiments where the separating layer provides protection the separating layer may also be referred to as a protective layer.

In some embodiments, the agent layer and the separating layer both comprise the same polymer. In other embodiments, the agent layer and the separating layer comprise different polymers.

As used herein, the term “preformed polymer” means a polymer which has already been at least partially polymerized before application, as opposed to a monomer or macromer which has not yet been polymerized and is polymerized as it is applied or after it is applied.

As used herein, term “(meth)acrylate” when used in describing polymers shall mean the form including the methyl group (methacrylate) or the form without the methyl group (acrylate).

Suitable polymers of the agent layer and/or separating layer include include polyaryl(meth)acrylates. Examples of polyaryl(meth)acrylates include poly-9-anthracenylmethacrylate, polychlorophenylacrylate, polymethacryloxy-2-hydroxybenzophenone, polymethacryloxybenzotriazole, polynaphthylacrylate, polynaphthylmethacrylate, poly-4-nitrophenylacrylate, polypentachloro(bromo, fluoro)(meth)acrylate, polyphenyl(meth)acrylate.

Suitable polymers of the agent layer and/or separating layer include polyaralkyl(meth)acrylates. Examples of polyaralkyl(meth)acrylates include polybenzyl(meth)acrylate, poly-2-phenethyl(meth)acrylate, poly-1-pyrenylmethylmethacrylate.

Suitable polymers of the agent layer and/or separating layer include polyaralkyl(meth)acrylates. Examples of polyaryloxyalkyl(meth)acrylates include polyphenoxyethyl(meth)acrylate, and polyethyleneglycolphenylether(meth)acrylates with varying polyethyleneglycol molecular weights.

Suitable polymers of the agent layer and/or separating layer also include polyalkyl(meth)acrylates. Examples of polyalkyl(meth)acrylate include polymethyl(meth)acrylate, polyethyl(meth)acrylate, polypropyl(meth)acrylate, polybutyl(meth)acrylate, and the like. In an embodiment, the polymer comprises polybutyl(meth)acrylate. In some embodiments, the polymer comprises a polyalkyl(meth)acrylate with an alkyl chain length from 2 to 8 carbons, and with average molecular weights from 50 kilodaltons to 900 kilodaltons. Unless otherwise indicated, all polymeric molecular weights described herein are “weight average” molecular weights (“Mw”).

Suitable polymers of the agent layer and/or separating layer also include poly(ethylene-co-vinyl acetate) (PEVA) having vinyl acetate concentrations of between about 8% and about 90%.

In some embodiments, the polymer of the agent layer and/or separating layer can be a combination of a first polymer and a second polymer. Examples of suitable first polymers include polyaryl(meth)acrylates, polyaralkyl(meth)acrylates, polyaralkyl(meth)acrylates, and polyalkyl(meth)acrylates, all as described above. Examples of suitable second polymers include poly(ethylene-co-vinyl acetate) (PEVA) as described above.

In an embodiment the polymer of the agent layer and/or separating layer includes mixtures of polyalkyl(meth)acrylates (e.g., polybutyl(meth)acrylate PBMA) or aromatic poly(meth)acrylates (e.g., polybenzyl(meth)acrylate) and poly(ethylene-co-vinyl acetate) copolymers (pEVA). An exemplary polymer mixture for use in this invention includes mixtures of poly(n-butyl methacrylate) (pBMA) and poly(ethylene-co-vinyl acetate) copolymers (pEVA). This mixture of polymers can be useful with absolute polymer concentrations (i.e., the total combined concentrations of both polymers in the coating composition), of between about 0.05 and about 70 wt. %, between about 0.25 and about 50 wt. %, or between about 0.25 and about 10 wt. %.

In an embodiment the polymer mixture of the agent layer and/or separating layer includes a first polymeric component with a weight average molecular weight of from about 50 kilodaltons to about 500 kilodaltons and a pEVA copolymer with a vinyl acetate content of from about 8 to about 90 weight percent, or between about 20 to about 40 weight percent. In a particular embodiment, the polymer mixture includes a first polymeric component with a molecular weight of from about 200 kilodaltons to about 400 kilodaltons and a pEVA copolymer with a vinyl acetate content of from about 30 to about 34 weight percent. In an embodiment the polymer mixture includes a polyalkyl(meth)acrylate (such as polybutyl(meth)acrylate) (pBMA)) with a weight average molecular weight of from about 100 kilodaltons to about 1000 kilodaltons and a pEVA copolymer with a vinyl acetate content of from about 20 to about 40 weight percent.

In an embodiment, first polymers can be (i) poly(alkylene-co-alkyl(meth)acrylates), (ii) ethylene copolymers with other alkylenes, (iii) polybutenes, (iv) diolefin derived non-aromatic polymers and copolymers, (v) aromatic group-containing copolymers, or (vi) epichlorohydrin-containing polymers.

Suitable poly(alkylene-co-alkyl(meth)acrylates) include those copolymers in which the alkyl groups are either linear or branched, and substituted or unsubstituted with non-interfering groups or atoms. Such alkyl groups can comprise from 1 to 8 carbon atoms, inclusive, and can comprise from 1 to 4 carbon atoms, inclusive. In an embodiment, the alkyl group is methyl. In some embodiments, copolymers that include such alkyl groups can comprise from about 15% to about 80% (wt) of alkyl acrylate. When the alkyl group is methyl, the polymer can contain from about 20% to about 40% methyl acrylate, or from about 25 to about 30% methyl acrylate. When the alkyl group is ethyl, the polymer can contain from about 15% to about 40% ethyl acrylate, and when the alkyl group is butyl, the polymer can contain from about 20% to about 40% butyl acrylate. The alkylene groups can be selected from ethylene and/or propylene. In an embodiment, the alkylene group is ethylene. The (meth)acrylate can comprise an acrylate (i.e., no methyl substitution on the acrylate group). Copolymers can provide a molecular weight (Mw) of about 50 kilodaltons to about 500 kilodaltons, or from about 50 kilodaltons to about 200 kilodaltons.

Copolymers such as poly(ethylene-co-methyl acrylate), poly(ethylene-co-butyl acrylate) and poly(ethylene-co-2-ethylhexyl acrylate) copolymers are available commercially from sources such as Atofina Chemicals, Inc., Philadelphia, Pa., and can be prepared using known methods.

With regard to suitable ethylene copolymers with other alkylenes, the alkylenes can be straight and branched alkylenes, as well as substituted or unsubstituted alkylenes. Examples include copolymers prepared from alkylenes that comprise from 3 to 8 branched or linear carbon atoms, inclusive, or alkylene groups that comprise from 3 to 4 branched or linear carbon atoms, inclusive. In an embodiment, the alkylene group contains 3 carbon atoms (e.g., propene). In an embodiment, the other alkylene is a straight chain alkylene (e.g., 1-alkylene).

Copolymers of this type can contain from about 20% to about 90% (based on moles) of ethylene, and more preferably, from about 35% to about 80% (mole) of ethylene. Such copolymers will have a molecular weight of between about 30 kilodaltons to about 500 kilodaltons. Examples include copolymers selected from the group consisting of poly(ethylene-co-propylene), poly(ethylene-co-1-butene), polyethylene-co-1-butene-co-1-hexene) and/or poly(ethylene-co-1-octene).

Examples of suitable copolymers include poly(ethylene-co-propylene) random copolymers in which the copolymer contains from about 35% to about 65% (mole) of ethylene; or from about 55% to about 65% (mole) ethylene, and the molecular weight of the copolymer is from about 50 kilodaltons to about 250 kilodaltons, or from about 100 kilodaltons to about 200 kilodaltons.

Copolymers of this type can optionally be provided in the form of random terpolymers prepared by the polymerization of both ethylene and propylene with one or more additional diene monomers, such as those selected from the group consisting of ethylidene norborane, dicyclopentadiene and/or hexadiene. Exemplary terpolymers of this type can include up to about 5% (mole) of the third diene monomer.

With respect to polybutenes, suitable examples include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In an embodiment, the polybutene contains at least about 90% (wt) of isobutylene or 1-butene. In an embodiment, the polybutene contains at least about 90% (wt) of isobutylene. The polybutene can contain non-interfering amounts of other ingredients or additives, for instance it can contain up to 1000 ppm of an antioxidant (e.g., 2,6-di-tert-butyl-methylphenol).

The polybutene can have a molecular weight between about 150 kilodaltons and about 1,000 kilodaltons, or between about 200 kilodaltons and about 600 kilodaltons. In an embodiment the polybutene has a molecular weight between about 350 kilodaltons and about 500 kilodaltons. Polybutenes having a molecular weight greater than about 600 kilodaltons, including greater than 1,000 kilodaltons are also available.

With respect to diolefin-derived, non-aromatic polymers and copolymers, examples include those in which the diolefin monomer used to prepare the polymer or copolymer is selected from butadiene (CH₂═CH—CH═CH₂) and/or isoprene (CH₂═CH—C(CH₃)═CH₂). For example, the polymer can be a homopolymer derived from diolefin monomers or is a copolymer of diolefin monomer with non-aromatic mono-olefin monomer, and optionally, the homopolymer or copolymer can be partially hydrogenated.

Such polymers can be selected from the group consisting of polybutadienes prepared by the polymerization of cis-, trans- and/or 1,2-monomer units, and more preferably a mixture of all three monomers, and polyisoprenes prepared by the polymerization of cis-1,4- and/or trans-1,4-monomer units.

Alternatively, the polymer is a copolymer, including graft copolymers, and random copolymers based on a non-aromatic mono-olefin monomer such as acrylonitrile, and an alkyl(meth)acrylate and/or isobutylene. Preferably, when the mono-olefin monomer is acrylonitrile, the interpolymerized acrylonitrile is present at up to about 50% by weight; and when the mono-olefin monomer is isobutylene, the diolefin is isoprene (e.g., to form what is commercially known as a “butyl rubber”). Exemplary polymers and copolymers have a Mw between about 150 kilodaltons and about 1,000 kilodaltons, or between about 200 kilodaltons and about 600 kilodaltons.

With respect to aromatic group-containing copolymers (including random copolymers, block copolymers and graft copolymers), examples include structures in which the aromatic group is incorporated into the copolymer via the polymerization of styrene. Examples also include structures in which the random copolymer is a copolymer derived from copolymerization of styrene monomer and one or more monomers selected from butadiene, isoprene, acrylonitrile, a C₁-C₄ alkyl(meth)acrylate (e.g., methyl methacrylate) and/or butene. Useful block copolymers include copolymer containing (a) blocks of polystyrene, (b) blocks of an polyolefin selected from polybutadiene, polyisoprene and/or polybutene (e.g., isobutylene), and (c) optionally a third monomer (e.g., ethylene) copolymerized in the polyolefin block.

The aromatic group-containing copolymers can contain about 10% to about 50% (wt) of polymerized aromatic monomer and the molecular weight of the copolymer can be from about 300 kilodaltons to about 500 kilodaltons. In an embodiment, the molecular weight of the copolymer can be from about 100 kilodaltons to about 300 kilodaltons.

Additional alternative first polymers include epichlorohydrin homopolymers and poly(epichlorohydrin-co-alkylene oxide) copolymers. For example, in the case of the copolymer, the copolymerized alkylene oxide is ethylene oxide. In an embodiment, epichlorohydrin content of the epichlorohydrin-containing polymer is from about 30% to 100% (wt), or from about 50% to 100% (wt). The epichlorohydrin-containing polymers can have a Mw from about 100 kilodaltons to about 300 kilodaltons.

When the first polymer includes (i) poly(alkylene-co-alkyl(meth)acrylates), (ii) ethylene copolymers with other alkylenes, (iii) polybutenes, (iv) diolefin derived non-aromatic polymers and copolymers, (v) aromatic group-containing copolymers, or (vi) epichlorohydrin-containing polymers, suitable second polymers include poly(alkyl(meth)acrylates) and poly(aromatic(meth)acrylates). In an embodiment, the second polymer is a polyalkyl(meth)acrylate. Examples of suitable poly(alkyl(meth)acrylates) include those with alkyl chain lengths from 2 to 8 carbons, inclusive, and with molecular weights from 50 kilodaltons to 900 kilodaltons. In an embodiment the polymer mixture includes a poly(alkyl(meth)acrylate) with a molecular weight of from about 100 kilodaltons to about 1000 kilodaltons, or from about 150 kilodaltons to about 500 kilodaltons. In an embodiment the polymer mixture includes a poly(alkyl(meth)acrylate) with a molecular weight of about 200 kilodaltons to about 400 kilodaltons. An example of a second polymer is poly(n-butyl methacrylate). Examples of other suitable polymers include poly(n-butyl methacrylate-co-methyl methacrylate), with a monomer ratio of 3:1, poly(n-butyl methacrylate-co-isobutyl methacrylate), with a monomer ratio of 1:1 and poly(t-butyl methacrylate). Such polymers are available commercially (e.g., from Sigma-Aldrich, Milwaukee, Wis.) with molecular weights ranging from about 150 kilodaltons to about 350 kilodaltons, and with varying inherent viscosities, solubilities and supplied forms (e.g., as slabs, granules, beads, crystals or powder).

When the first polymer includes (i) poly(alkylene-co-alkyl(meth)acrylates), (ii) ethylene copolymers with other alkylenes, (iii) polybutenes, (iv) diolefin derived non-aromatic polymers and copolymers, (v) aromatic group-containing copolymers, or (vi) epichlorohydrin-containing polymers, suitable poly(aromatic(meth)acrylates) include poly(aryl(meth)acrylates), poly(aralkyl(meth)acrylates), poly(alkaryl(meth)acrylates), poly(aryloxyalkyl(meth)acrylates), and poly(alkoxyaryl(meth)acrylates). A poly(aralkyl(meth)acrylate) can be made from aromatic esters derived from alcohols also containing aromatic moieties, such as benzyl alcohol. A poly(alkaryl(meth)acrylate) can be made from aromatic esters derived from aromatic alcohols such as p-anisole. Suitable poly(aromatic(meth)acrylates) include aryl groups having from 6 to 16 carbon atoms and with molecular weights from about 50 to about 900 kilodaltons. Examples of suitable poly(aryl(meth)acrylates) include poly(9-anthracenyl methacrylate), poly(chlorophenyl acrylate), poly(methacryloxy-2-hydroxybenzophenone), poly(methacryloxybenzotriazole), poly(naphthyl acrylate), poly(naphthyl methacrylate), poly-4-nitrophenylacrylate, poly(pentachloro(bromo, fluoro)acrylate) and methacrylate, poly(phenyl acrylate) and poly(phenyl methacrylate). Examples of suitable poly(aralkyl (meth)acrylates) include poly(benzyl acrylate), poly(benzyl methacrylate), poly(2-phenethyl acrylate), poly(2-phenethyl methacrylate) and poly(1-pyrenylmethyl methacrylate). Examples of suitable poly(alkaryl(meth)acrylates include poly(4-sec-butylphenyl methacrylate), poly(3-ethylphenyl acrylate), and poly(2-methyl-1-naphthyl methacrylate). Examples of suitable poly(aryloxyalkyl(meth)acrylates) include poly(phenoxyethyl acrylate), poly(phenoxyethyl methacrylate), and poly(polyethylene glycol phenyl ether acrylate) and poly(polyethylene glycol phenyl ether methacrylate) with varying polyethylene glycol molecular weights. Examples of suitable poly(alkoxyaryl(meth)acrylates) include poly(4-methoxyphenyl methacrylate), poly(2-ethoxyphenyl acrylate) and poly(2-methoxynaphthyl acrylate).

When the first polymer includes (i) poly(alkylene-co-alkyl(meth)acrylates), (ii) ethylene copolymers with other alkylenes, (iii) polybutenes, (iv) diolefin derived non-aromatic polymers and copolymers, (v) aromatic group-containing copolymers, or (vi) epichlorohydrin-containing polymers, and the second polymers is one of poly(alkyl(meth)acrylates) and poly(aromatic(meth)acrylates), absolute polymer concentrations (i.e., the total combined concentrations of both polymers in the coating composition), can be about 0.1 and about 50 percent (by weight), or about 0.1 and about 8 percent (by weight). In an embodiment, the polymer mixtures can contain at least about 10 percent by weight of either the first polymer or the second polymer.

In an embodiment the polymer mixture includes a first polymer component comprising one or more polymers selected from the group consisting of (i) poly(alkylene-co-alkyl(meth)acrylates, (ii) ethylene copolymers with other alkylenes, (iii) polybutenes, (v) diolefin derived non-aromatic polymers and copolymers, (vi) aromatic group-containing copolymers, and (vi) epichlorohydrin-containing polymers, and a second polymer component selected from the group consisting of poly(alkyl(meth)acrylates) and poly(aromatic(meth)acrylates) and having a molecular weight of preferably from about 150 kilodaltons to about 500 kilodaltons, or about 200 kilodaltons to about 400 kilodaltons.

Acrylate or methacrylate monomers or polymers and/or their parent alcohols are commercially available from Sigma-Aldrich (Milwaukee, Wis.) or from Polysciences, Inc, (Warrington, Pa.).

Preformed polymers of the invention are typically applied as a coating composition. When used to form the agent layer, a coating composition can be prepared to include a solvent, a combination of complementary polymers dissolved in the solvent, and the active agent or agents dispersed in the polymer/solvent mixture. The pharmaceutical agent itself can either be soluble in the solvent or form a dispersion throughout the solvent. When used to form the separating layer, a coating composition can be prepared to include a solvent and a combination of complementary polymers dissolved in the solvent. The solvent can include alcohols (e.g., methanol, butanol, propanol and isopropanol), alkanes (e.g., halogenated or unhalogenated alkanes such as chloroform, hexane, and cyclohexane), amides (e.g., dimethylformamide), ethers (e.g., THF and dioxolane), ketones (e.g., methylethylketone), aromatic compounds (e.g., toluene and xylene), nitriles (e.g., acetonitrile) and esters (e.g., ethyl acetate).

The resultant composition can be applied to the device in any suitable fashion. By way of example, it can be applied directly to the surface of a device, or alternatively, to the surface of a surface-modified device, by dipping, spraying, or any conventional technique. In some embodiments, the composition may be deposited under conditions of controlled relative humidity. The composition can be coated onto a device surface in one or more applications. The method of applying the coating composition to the device is typically governed by the geometry of the device and other process considerations. The coating is subsequently cured by evaporation of the solvent. The curing process can be performed at room temperature, elevated temperature, or with the assistance of vacuum.

Polymers of the Elution Control Layer

In some embodiments of the invention the elution control layer can include a vapor and/or plasma deposited polymer. In an embodiment, the vapor and/or plasma deposited polymers include parylene and parylene derivatives. In some embodiments, the layer of vapor and/or plasma deposited polymer can be about 0.01 to 10.0 microns thick. In an embodiment, the layer of vapor and/or plasma deposited polymer can be about 0.05 to 2.0 microns thick.

“Parylene” is both a generic name for a known group of polymers based on p-xylylene and made by vapor or plasma phase polymerization, and a name for the unsubstituted form of the polymer; the latter usage is employed herein for the term “parylene”. The term “parylene derivative” will refer to the known group of polymers based on p-xylylene and made by vapor or plasma phase polymerization. Common parylene derivatives include parylene-C (see FIG. 9a), parylene-N (see FIG. 9b), and parylene-D (see FIG. 9c).

In an embodiment, the elution control layer includes at least one of poly 2-chloro-paraxylylene (parylene C), polyparaxylylene (parylene N), poly 2,5-dichloro-paraxylylene (parylene D). In some embodiments, the elution control layer includes poly 2-chloro-paraxylylene (parylene C). In some embodiments, the elution control layer includes poly 2,3,5,6-tetrafluoro-paraxylylene.

In an embodiment, the polymer of the elution control layer includes mono-, di-, tri-, and tetra-halo substituted polyparaxylylene. In an embodiment, the polymer includes mono-, di-, tri-, and tetra-chloro substituted polyparaxylylene. In an embodiment, the polymer includes mono-, di-, tri-, and tetra-fluoro substituted polyparaxylylene.

Parylene or a parylene derivative can be created by first heating p-xylene or a suitable derivative at an appropriate temperature (for example, at about 950° C.) to produce the cyclic dimer di-p-xylylene (or a derivative thereof). The resultant solid can be separated in pure form, and then cracked and pyrolyzed at an appropriate temperature (for example, at about 680° C.) to produce a monomer vapor of p-xylylene (or derivative); the monomer vapor is cooled to a suitable temperature (for example, below 50° C.) and allowed to condense on the desired object, for example, on the separating layer. Because parylene does not require a separate iniator, it can be referred to as a self-initiating polymer. An unsubstituted parylene polymer can have the repeating structure -(p-CH₂—C₆H₄—CH₂)ₙ-, with n equal to about 5,000 daltons, and a molecular weight of about 500,000 daltons.

Parylene and parylene derivative coatings applicable by vapor deposition are commercially available from or through a variety of sources, including Specialty Coating Systems (100 Deposition Drive, Clear Lake, Wis. 54005), Para Tech Coating, Inc. (35 Argonaut, Aliso Viejo, Calif. 92656) and Advanced Surface Technology, Inc. (9 Linnel Circle, Billerica, Mass. 01821-3902).

The plasma deposition process can also be used to deposit polymers such as poly(ethylene oxide), poly(ethylene glycol), and poly(propylene oxide), as well as polymers of silicone, methane, tetrafluoroethylene (including TEFLON® brand polymers), tetramethyldisiloxane, and others. Accordingly, the term “vapor and/or plasma deposited polymer” includes more than just parylene and parylene derivatives.

Substrates

Embodiments of the invention provide the ability to deliver active agents from a variety of substrate surfaces including metals, polymers, ceramics, and natural materials.

Metals include, but are not limited to, titanium, stainless steel, and cobalt chromium. Suitable metals can also include the noble metals such as gold, silver, copper, and platinum. Finally, suitable metals can include alloys such as nitinol or cobalt chromium alloys.

Polymers include those formed of synthetic polymers, including oligomers, homopolymers, and copolymers resulting from either addition or condensation polymerizations. Examples include, but not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, styrene, vinyl chloride, vinyl acetate, vinyl pyrrolidone, and vinylidene difluoride, condensation polymers including, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polylactic acid, polyglycolic acid, polydimethylsiloxanes, and polyetheretherketone.

Embodiments of the invention can also include the use of ceramics as a substrate. The ceramics include, but are not limited to, silicon nitride, silicon carbide, zirconia, and alumina, as well as glass, silica, and sapphire.

Certain natural materials are also suitable including human tissue, when used as a component of a device, such as bone, cartilage, skin and teeth; and other organic materials such as wood, cellulose, compressed carbon, rubber, silk, wool, and cotton.

The composition of the substrate can also include resins, polysaccharides, silicon, or silica-based materials, glass, films, gels, and membranes.

Devices

Embodiments of the invention can be used with many different types of medical devices. These devices can include both implantable devices and non-implantable medical devices.

Embodiments of the invention can be used with implantable, or transitorily implantable, devices including, but not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), catheters (including arterial, intravenous, blood pressure, stent graft, etc.), valves (e.g., polymeric or carbon mechanical valves, tissue valves, valve designs including percutaneous, sewing cuff, and the like), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuroaneurysm treatment coils, and neuropatches; ear nose and throat devices such as nasal buttons, nasal and airway splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; biosensor devices including glucose sensors, cardiac sensors, intra-arterial blood gas sensors; oncological implants; and pain management implants.

Classes of suitable non-implantable devices can include dialysis devices and associated tubing, catheters, membranes, and grafts; autotransfusion devices; vascular and surgical devices including atherectomy catheters, angiographic catheters, intraaortic balloon pumps, intracardiac suction devices, blood pumps, blood oxygenator devices (including tubing and membranes), blood filters, blood temperature monitors, hemoperfusion units, plasmapheresis units, transition sheaths, dialators, intrauterine pressure devices, clot extraction catheters, percutaneous transluminal angioplasty catheters, electrophysiology catheters, breathing circuit connectors, stylets (vascular and non-vascular), coronary guide wires, peripheral guide wires; dialators (e.g., urinary, etc.); surgical instruments (e.g. scalpels and the like); endoscopic devices (such as endoscopic surgical tissue extractors, esophageal stethoscopes); and general medical and medically related devices including blood storage bags, umbilical tape, membranes, gloves, surgical drapes, wound dressings, wound management devices, needles, percutaneous closure devices, transducer protectors, pessary, uterine bleeding patches, PAP brushes, clamps (including bulldog clamps), cannulae, cell culture devices, materials for in vitro diagnostics, chromatographic support materials, infection control devices, colostomy bag attachment devices, birth control devices; disposable temperature probes; and pledgets.

Coatings of the invention can also be applied to devices other than medical devices. By way of example, coatings which elute agents that control the growth of biological organisms can be useful in a variety of contexts such as water delivery pipes, boat hulls, flumes, tanks, structures designed to be at least partially submerged, structures subject to biofilm formation, and the like.

Active Agents

As used herein, the term “active agent” means a compound that has a particular desired activity. For example, an active agent can be a therapeutic compound that exerts a specific activity on a subject. In some embodiments, active agent will, in turn, refer to a peptide, protein, carbohydrate, nucleic acid, lipid, polysaccharide or combinations thereof, or synthetic inorganic or organic molecule, that causes a desired biological effect when administered in vivo to an animal, including but not limited to birds and mammals, including humans.

Active agents useful in the present invention can include many types of therapeutics including thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, antimicrobial agents, antibiotics, antibacterial agents, antiparasite and/or antiprotozoal solutes, antiseptics, antifungals, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, anti-proliferatives, anticancer chemotherapeutic agents, anti-neoplastic agents, antipolymerases, antivirals, anti-AIDS substances, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, extracellular matrix components, ACE inhibitors, free radical scavengers, chelators, anti-oxidants, photodynamic therapy agents, gene therapy agents, anesthetics, immunotoxins, neurotoxins, opioids, dopamine agonists, hypnotics, antihistamines, tranquilizers, anticonvulsants, muscle relaxants and anti-Parkinson substances, antispasmodics and muscle contractants, anticholinergics, ophthalmic agents, antiglaucoma solutes, prostaglandins, antidepressants, antipsychotic substances, neurotransmitters, anti-emetics, imaging agents, specific targeting agents, and cell response modifiers. A more complete listing of classes of medicaments may be found in the Pharmazeutische Wirkstoffe, ed. A. Von Kleemann and J. Engel, Georg Thieme Verlag, Stuttgart/New York, 1987, incorporated herein by reference.

More specifically, in embodiments the active agent can include heparin, covalent heparin, synthetic heparin salts, or another thrombin inhibitor; hirudin, hirulog, argatroban, D-phenylalanyl-L-poly-L-arginyl chloromethyl ketone, or another antithrombogenic agent; urokinase, streptokinase, a tissue plasminogen activator, or another thrombolytic agent; a fibrinolytic agent; a vasospasm inhibitor; a calcium channel blocker, a nitrate, nitric oxide, a nitric oxide promoter, nitric oxide donors, dipyridamole, or another vasodilator; HYTRIN® or other antihypertensive agents; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; dimethyl sulfoxide (DMSO), a retinoid, or another antisecretory agent; cytochalasin or another actin inhibitor; cell cycle inhibitors; remodeling inhibitors; deoxyribonucleic acid, an antisense nucleotide, or another agent for molecular genetic intervention; methotrexate, or another antimetabolite or antiproliferative agent; tamoxifen citrate, TAXOL®, paclitaxel, or the derivatives thereof, rapamycin, vinblastine, vincristine, vinorelbine, etoposide, tenopiside, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, chlorambucil, ethylenimines, methylmelamines, alkyl sulfonates (e.g., busulfan), nitrosoureas (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, or other anti-cancer chemotherapeutic agents; cyclosporin, tacrolimus (FK-506), azathioprine, mycophenolate mofetil, mTOR inhibitors, or another immunosuppressive agent; cortisol, cortisone, dexamethasone, dexamethasone sodium phosphate, dexamethasone acetate, dexamethasone derivatives, betamethasone, fludrocortisone, prednisone, prednisolone, 6U-methylprednisolone, triamcinolone, or another steroidal agent; trapidil (a PDGF antagonist), angiopeptin (a growth hormone antagonist), angiogenin, a growth factor (such as vascular endothelial growth factor (VEGF)), or an anti-growth factor antibody, or another growth factor antagonist or agonist; dopamine, bromocriptine mesylate, pergolide mesylate, or another dopamine agonist; ⁶⁰Co (5.3 year half life), ¹⁹²Ir (73.8 days), ³²P (14.3 days), ¹¹¹In (68 hours), ⁹⁰Y (64 hours), ⁹⁹Tc (6 hours), or another radiotherapeutic agent; iodine-containing compounds, barium-containing compounds, gold, tantalum, platinum, tungsten or another heavy metal functioning as a radiopaque agent; a peptide, a protein, an extracellular matrix component, a cellular component or another biologic agent; captopril, enalapril or another angiotensin converting enzyme (ACE) inhibitor; angiotensin receptor blockers; enzyme inhibitors (including growth factor signal transduction kinase inhibitors); ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹I-, ³²P- or ³⁶S-radiolabelled form or other radiolabelled form of any of the foregoing; estrogen or another sex hormone; AZT or other antipolymerases; acyclovir, famciclovir, rimantadine hydrochloride, ganciclovir sodium, Norvir, Crixivan, or other antiviral agents; 5-aminolevulinic acid, metatetrahydroxyphenylchlorin, hexadecafluorozinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine betahydroxylase conjugated to saporin, or other antibody targeted therapy agents; gene therapy agents; enalapril and other prodrugs; PROSCAR®, HYTRIN® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these. A comprehensive listing of bioactive agents can be found in The Merck Index, Thirteenth Edition, Merck & Co. (2001).

Antibiotics are substances which inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, erythromycin and cephalosporins. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Antiseptics are recognized as substances that prevent or arrest the growth or action of microorganisms, generally in a nonspecific fashion, e.g., either by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Antiviral agents are substances capable of destroying or suppressing the replication of viruses. Examples of anti-viral agents include α-methyl-1-adamantanemethylamine, hydroxy-ethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatecho-1, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Local anesthetics are substances that have an anesthetic effect in a localized region. Examples of such anesthetics include procaine, lidocaine, tetracaine and dibucaine.

Imaging agents are agents capable of imaging a desired site, e.g., tumor, in vivo. Examples of imaging agents include substances having a label that is detectable in vivo, e.g., antibodies attached to fluorescent labels. The term antibody includes whole antibodies or fragments thereof.

Cell response modifiers are chemotactic factors such as platelet-derived growth factor (PDGF). Other chemotactic factors include neutrophil-activating protein, monocyte chemoattractant protein, macrophage-inflammatory protein, SIS (small inducible secreted), platelet factor, platelet basic protein, melanoma growth stimulating activity, epidermal growth factor, transforming growth factor alpha, fibroblast growth factor, platelet-derived endothelial cell growth factor, insulin-like growth factor, nerve growth factor, bone growth/cartilage-inducing factor (alpha and beta), and matrix metalloproteinase inhibitors. Other cell response modifiers are the interleukins, interleukin receptors, interleukin inhibitors, interferons, including alpha, beta, and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins, antisense molecules, androgenic receptor blockers and statin agents.

In some embodiments, the active agent can include therapeutic agents that are hydrophilic. In some embodiments, the active agent can include therapeutic agents that are small hydrophilic agents. As used herein, small hydrophilic agents include those with a molecular weight of less than 5 kilodaltons and with water solubility of greater than 10 mg/mL at 25 degrees Celsius. In some embodiments, small hydrophilic agents can include those with a water solubility of greater than 100 mg/ml at 25 degrees Celsius. By way of example, small hydrophilic agents can include Trigonelline HCL, diclofenac, and chlorhexidine diacetate. Small hydrophilic agents can include organic salts or other charged molecules. Small hydrophilic agents can also include those that are non-ionic and incorporate other hydrophilic groups.

In some embodiments, the active agent can include therapeutic agents that are relatively hydrophobic. In some embodiments, the active agent can include therapeutic agents that are small hydrophobic agents. As used herein, small hydrophobic agents can include those with a molecular weight of less than 5 kilodaltons and with water solubility of less than about 10 mg/mL at 25 degrees Celsius. By way of example, small hydrophobic agents can include anti-inflammatory agents, such as steroidal anti-inflammatory agents. Small hydrophobic agents can include paclitaxel, camptothecin, doxorubicin, cisplatin, 5-fluorouracil, cyclosporine A, amphotericin B, itraconazole, ketoconazole, indomethacin, testosterone, estradiol, dexamethasone, prednisolone, and triamcinolone acetonide. In an embodiment, the small hydrophobic agent can include dexamethasone.

In an embodiment, the active agent can be in a microparticle. In an embodiment, microparticles can be dispersed on the surface of the substrate.

The weight of the coating attributable to the bioactive agent can be in the range of about 1 microgram to about 10 milligrams of bioactive agent per cm² of the effective surface area of the device. By “effective” surface area it is meant the surface amenable to being coated with the composition itself. For a flat, nonporous, surface, for instance, this will generally be the macroscopic surface area itself, while for considerably more porous or convoluted (e.g., corrugated, pleated, or fibrous) surfaces the effective surface area can be significantly greater than the corresponding macroscopic surface area. In an embodiment, the weight of the coating attributable to the bioactive is between about 0.01 mg and about 0.5 mg of bioactive agent per cm² of the gross surface area of the device.

Embodiments of the invention will now be described through examples. However, one of skill in the art will appreciate that these are only examples and not serve to limit the scope of the invention.

### EXAMPLE 1

### Controlled Release of a Model Hydrophilic Active Agent

A model hydrophilic drug, Trigonelline HCl (“Trig”), was obtained from Aldrich (product number S414255). Stainless steel stents were obtained from Laserage Technology Corporation, Waukegan, Ill. Two solutions were prepared for deposition of the Trig onto the stents. The first solution was a base layer solution and was comprised of 15 wt. % Trig and 85 wt. % of a polymer dissolved in a solvent mixture of 85% THF/15% MeOH (by volume) to result in a solution having a total solids concentration of 30 mg/ml. The second solution was a protective layer solution and was comprised of poly n-butyl methacrylate (PBMA) dissolved in THF at a solids concentration of 20 mg/ml. The base layer solution was applied to the stents using an ultra sonic spray deposition method in amounts as shown in Table 1. The protective layer solution was applied to the stents using an ultra sonic spray deposition method in amounts as shown in Table 1.

Next, an elution control layer, in this case a layer of Parylene C (Specialty Coating Systems), was deposited onto the protective layer using a vapor deposition process. The coated stents were suspended with stainless steel wire in the vapor deposition chamber (Specialty Coating Systems PDS 2010). Various weights of Parylene C dimer (between 0.5 and 2.5 grams) were individually placed into the vapor deposition chamber furnace. The vapor deposition chamber repeatedly cycled between four steps in the vapor deposition process: 1) evacuation of the chamber; 2) vaporization of the Parylene C dimer; 3) intense heating of the Parylene C dimer to produce reactive monomer; 4) deposition of the reactive Parylene C monomer onto the protective layer substrate and subsequent formation of a cross-linked, outer layer in amounts as shown in Table 1.

TABLE 1

| | Base Layer: | Protective Layer: | Elution Control |
|---|---|---|---|
| | (polymer wt./ | (preformed polymer | Layer: |
| Sample | active agent wt.) | wt.) | (Parylene C wt.) |
| A | 1576 μg/236 μg | 545 μg | 0 μg |
| B | 1503 μg/225 μg | 549 μg | 115 μg |
| C | 1574 μg/236 μg | 559 μg | 315 μg |
| D | 1557 μg/234 μg | 538 μg | 487 μg |
| E | 1541 μg/231 μg | 251 μg | 584 μg |
| F | 1545 μg/232 μg | 248 μg | 770 μg |

In vitro drug elution studies were completed to study the elution rate and recovery of Trig from the coated stents. The stents were placed into vials containing 4 ml of phosphate buffered saline (PBS). The PBS in each vial was stirred using a Variomag electronic stirrer and the vials were kept in a water bath at a constant 37 degrees Celsius temperature. At various time points over 11 days the stents were placed into new vials containing a fresh 4 ml of PBS. The drug-containing 4 ml PBS samples were then analyzed for Trig by measuring absorbance at 265 nm using a UV spectrophotometer (Shimadzu UV-1601). The Trig absorbances were converted into amounts of Trig using a calibration curve and plotted versus time.

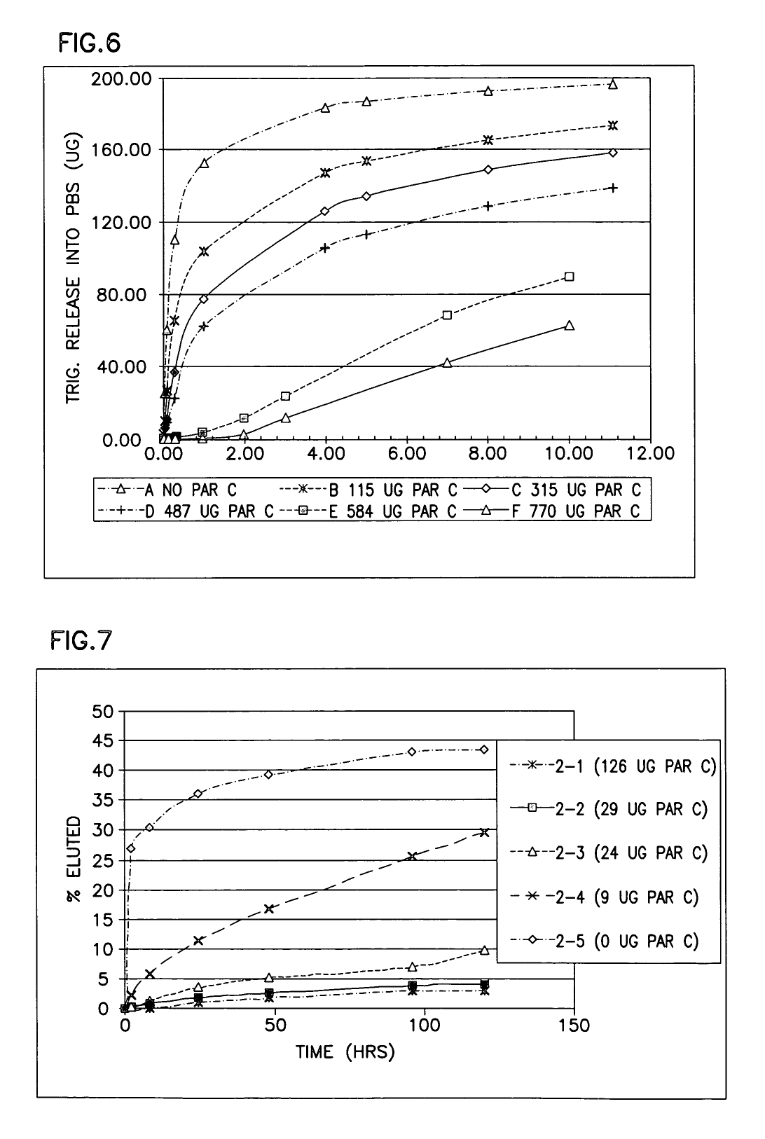

The results are shown in FIG. 6. The results show that an elution control layer over a protective layer provides control over the elution profile for an exemplary hydrophilic active agent. Specifically, sample A (no parylene) shows a very rapid elution profile and demonstrates that a PBMA coat alone is insufficient to provide control over the elution profile of some active agents. In contrast, samples B, C, D, E, and F show that the elution profile can be manipulated as desired by varying the amount of the elution control layer deposited. By way of example, a reverse-burst elution profile can be generated, as shown in samples E and F.

After completion of the elution testing, the stents were removed from the vials, lightly rinsed with deionized water, and dried under vacuum for one night. The stents were placed in vials with 10 mL of chloroform and agitated to dissolve the remaining coating. Next 5 ml of deionized water was added to the chloroform and the vials were vigorously agitated for 30 seconds. The vials were then allowed to sit undisturbed for several hours while the water and chloroform separated into two distinct phases, with the dissolved coating layers in the bottom chloroform phase and the Trig drug in the upper aqueous phase. The upper aqueous phase was then sampled and the Trig absorbance was read using the UV absorbance in the manner stated above. The total amount of Trig recovered in this elution experiment was then compared to the theoretical Trig loading as shown in Table 2 below.

As shown in Table 2 below, the average total recovery of Trig drug from the multi-layer coating matrix is 106%, well within the experimental error for the detection method used. Therefore, the Trig loaded in the multi-layer coating was free to elute out of the coating, and was not chemically bound to the Parylene C or degraded by the presence of free radicals during the Parylene C deposition process.

TABLE 2

| Sample | Drug Recovered - | Drug Recovered - | |
|---|---|---|---|
| Number | Elution | Extraction | Total Recovered |
| B | 173 μg (77%) | 74 μg (33%) | 247 μg (109%) |
| C | 158 μg (67%) | 79 μg (34%) | 237 μg (100%) |
| D | 139 μg (59%) | 117 μg (50%) | 256 μg (109%) |

### EXAMPLE 2

### Controlled Release of Model Drug Dexamethasone

The release of a model hydrophobic drug Dexamethasone (“Dex”) was examined. Dex can be considered a hydrophobic drug as it has a water solubility of 0.089 mg/ml. Dex was obtained from Sigma (product number D1756). Two separate coating solutions were prepared. In the first Dex coating solution (DEX1), 33 wt. % Dex was combined with 33 wt. % polybutadiene (PBD) and 33 wt. % polybutylmethacrylate (PBMA) in THF to result in a solution with a total solids concentration of 30 mg/ml. In the second Dex coating solution (DEX2), 30 wt. % Dex was combined with 35 wt. % PBD and 35 wt. % PBMA in THF to result in a solution with a total solids concentration of 30 mg/ml. These solutions were applied to stainless steel stents (Laserage Technology Corporation, Waukegan, Ill.) using an ultrasonic spray deposition method to create base composition layers. Specifically, DEX1 was applied to three stents (2-2, 2-3, and 2-4) and DEX2 was applied to two stents (2-1 and 2-5). The stents were dried at least one night under ambient conditions and then were weighed with a microbalance (Mettler Toledo UMT2) to obtain the base layer coating weight (see table 4 below).

Next, a protective layer solution was prepared. This solution was comprised of PBMA at 20 mg/mL (wt./vol) in THF. The protective layer solution was applied over the base composition using the same ultrasonic deposition method. The stents were dried under ambient conditions and were weighed with a microbalance to obtain the protective layer coating weight (see table 4 below).

An elution control layer, in this case Parylene C (Specialty Coating Systems), was then deposited onto the protective layer using a vapor deposition process. The coated stents were suspended with stainless steel wire in the vapor deposition chamber (Specialty Coating Systems PDS 2010). Various weights of Parylene C dimer (between 0.1 and 2.0 grams) were individually placed into the vapor deposition chamber furnace. The vapor deposition chamber repeatedly cycled between four steps in the vapor deposition process: 1) evacuation of the chamber 2) vaporization of the Parylene C dimer; 3) intense heating of the Parylene C dimer to produce reactive monomer; 4) deposition of the reactive Parylene C monomer onto the protective layer substrate and subsequent formation of a cross-linked, elution-control layer. The stents were weighed with a microbalance to obtain the elution control layer coating weight (see table 4 below).

TABLE 4

| | Base Layer: | | Elution Control |
|---|---|---|---|
| | (polymer wt./ | Protective Layer: | Layer: |
| | active agent wt.) | Preformed polymer | Parylene C wt. |
| Sample # | (ug/ug) | wt. (ug) | (ug) |
| 2-1 | 602/181 | 104 | 126 |
| 2-2 | 653/215 | 75 | 29 |
| 2-3 | 622/205 | 107 | 24 |
| 2-4 | 622/205 | 104 | 9 |
| 2-5 | 603/181 | 116 | 0 |

In vitro drug elution studies were completed to study the elution rate of Dex from the coated stents. The stents were placed into continuous flow tubes containing 35 mL of deionized water with 2 wt. % sodium laurel sulfate (SLS). The media in each tube was circulated at 16 ml/min and was held at a constant 37° C. temperature. At various time points over a period of approximately five days the tubes were automatically analyzed for Dex UV absorbance at 242 nm using a UV spectrophotometer. The Dex absorbances were converted into amounts of Dex using an absorbance versus concentration calibration curve and plotted versus time (see FIG. 7). The data show that elution rate for a relatively hydrophobic active agent decreased with increasing amounts of the elution control layer, in this case parylene.

### EXAMPLE 3

### Controlled Release of a Model Hydrophilic Drug Mimic

The release of a model hydrophilic drug mimic, 1,4 Dimethyl pyridinium iodide (DMPI) was examined. DMPI was obtained from Aldrich (product number 37,643-4). DMPI was prepared in a base layer coating solution. This base layer coating solution was comprised of 15 wt. % DMPI and 85 wt. % of a polymer dissolved at a total solids concentration of 30 mg/mL (wt./vol) in 85/15 (vol/vol) THF/MeOH. This solution was applied to stainless steel stents (Laserage Technology Corporation, Waukegan, Ill.) using an ultra sonic spray deposition method to create a base composition layer. The stents were dried under vacuum and then weighed with a microbalance (Mettler Toledo UMT2) to obtain the base layer coating weight (shown in Table 5 below).

Next, a protective layer solution was prepared. This solution was comprised of polybutylmethacrylate (PBMA) at 20 mg/mL (wt./vol) in THF. The protective layer solution was applied over the base layer using the same ultra sonic deposition method. The stents were dried under vacuum and were weighed with a microbalance to obtain the protective layer coating weight (shown in Table 5 below).

An elution control layer, Parylene C (Specialty Coating Systems), was then deposited onto the protective layer using a vapor deposition process. The coated stents were suspended with stainless steel wire in the vapor deposition chamber (Specialty Coating Systems PDS 2010). Various weights of Parylene C dimer (between 0.1 and 2.5 grams) were individually placed into the vapor deposition chamber furnace. The vapor deposition chamber repeatedly cycled between four steps in the vapor deposition process: 1) evacuation of the chamber 2) vaporization of the Parylene C dimer; 3) intense heating of the Parylene C dimer to produce reactive monomer; 4) deposition of the reactive Parylene C monomer onto the protective layer substrate and subsequent formation of a cross-linked, elution-control layer. The stents were again weighed with a microbalance to obtain the elution-control layer weight (shown in Table 5 below).

TABLE 5

| | Base Layer: | | Elution Control |
|---|---|---|---|
| | (polymer wt./ | Protective Layer: | Layer: |
| | active agent wt.) | Preformed polymer | Parylene C wt. |
| Sample # | (ug/ug) | wt. (ug) | (ug) |
| 1 | 1669/250 | 0 | 0 |
| 2 | 1612/242 | 1347 | 0 |
| 3 | 1524/229 | 1340 | 72 |
| 4 | 1484/223 | 1351 | 199 |

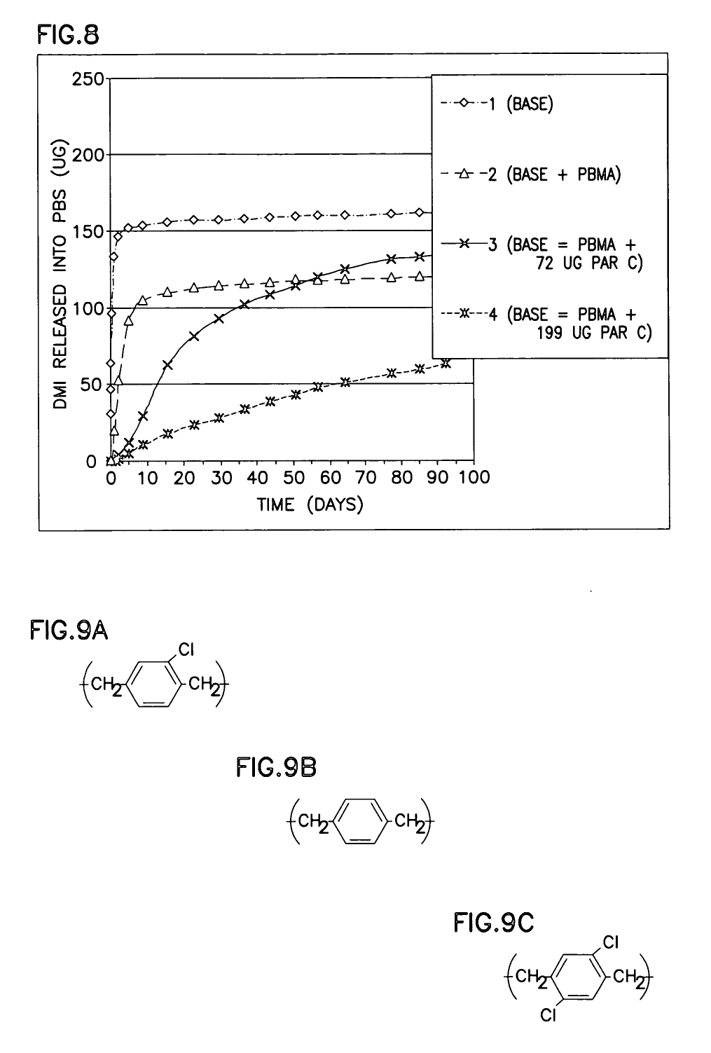

In vitro drug elution studies were completed to study the elution rate and recovery of DMPI from the coated stents. The stents were placed into vials containing 4 mL of phosphate buffered saline (PBS). The PBS in each vial was stirred using a Variomag electronic stirrer and the vials were kept in a water bath at a constant 37° C. temperature. At various time points over a period of approximately 90 days the vials were sampled and the stents were placed into new vials containing a fresh 4 mL of PBS. The drug-containing PBS samples were then analyzed for DMPI absorbance at 224 nm using a UV spectrophotometer (Shimadzu UV-1601). The DMPI absorbances were converted into amounts of DMPI using an absorbance versus concentration calibration curve and plotted versus time (see FIG. 8). The data show that a PBMA coat alone (sample 2) is insufficient to provide control over the elution profile of some active agents, such as small hydrophilic active agents. In contrast, samples 3 and 4 show that the elution profile can be manipulated as desired by varying the amount of elution control layer deposited.

### EXAMPLE 4

### Durability Test

Stents coated by means as described above in Example 1 are placed onto stent delivery balloon catheters and crimped down tightly using a hand crimper (Machine Solutions, Brooklyn, N.Y.). The crimped stent and balloon are placed into a vial containing 4 ml of deionized water. The vial is kept in a water bath at a constant 37 degrees Celsius temperature. The stent and balloon are soaked in the deionized water for five minutes, and then the balloon is expanded to 7 atmospheres using a hand pump. The stent and balloon are removed from the vial and the balloon is deflated back to its original size. The stent is air dried for several minutes and then is examined with a stereo microscope under 30-40 times magnification for the presence of mechanical defects in the coating layers such as tearing, cracking, or delamination.

### EXAMPLE 5

### Flexibility Test

A suitable flexibility test, in turn, can be used to detect imperfections (when examined by scanning electron microscopy “SEM”) that develop in the course of flexing of a coated specimen, an in particular, signs of cracking at or near the area of a bend.

One end of a specimen (1.0 cm) is clamped in a bench vice. The free end of the specimen (1.0 cm) is held with a pliers. The wire is bent until the angle it forms with itself is less than 90 degrees. The wire is removed from the vice and examined by SEM to determine the effect of the bending on the coating.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration to. The phrase “adapted and configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains. The invention has been described with reference to various specific and preferred embodiments and techniques. However, it should be understood that many variations and modifications may be made while remaining within the spirit and scope of the invention.

1. A medical device comprising:
a structure configured for introduction into a subject;
a base composition disposed on the structure comprising an active agent;
a first layer disposed on the base composition, the first layer comprising a first polymer and configured to separate the base composition from a second layer; and
the second layer disposed on the first layer, the second layer comprising a second polymer and configured to provide controlled release of the active agent through the second layer; wherein the second layer has release characteristics that are distinct from the first layer.

2. The medical device of claim 1, comprising an implantable device.

3. The medical device of claim 1, wherein the active agent is hydrophilic.

4. The medical device of claim 1, wherein the active agent has a molecular weight of less than 5 kilodaltons and has a water solubility of greater than 10 mg/mL at 25 degrees Celsius.

5. The medical device of claim 1, the first layer comprising a polyalkyl(meth)acrylate.

6. The medical device of claim 1, the first layer comprising poly(n-butyl methacrylate) and poly(ethylene-co-vinyl acetate).

7. The medical device of claim 1, the second layer comprising a plasma or vapor deposited polymer.

8. The medical device of claim 1, the second layer comprising at least one of poly 2-chloro-paraxylylene (parylene C), polyparaxylylene (parylene N), poly 2,5-dichloro-paraxylylene (parylene D).

9. The medical device of claim 1, the second layer comprising poly 2-chloro-paraxylylene (parylene C).

10. The medical device of claim 1, wherein the first layer adheres to the base composition.

11. The medical device of claim 1, wherein the first layer is configured to protect the active agent.

12. The medical device of claim 1, wherein the second layer is selected to produce controlled release comprising a reverse-burst elution profile.

13. A coating comprising:
a base composition comprising an active agent;
a first layer comprising a first polymer disposed on the base composition, the first layer configured to separate the base composition from a second layer; and
the second layer comprising a second polymer and disposed on the first layer, the second layer configured to provide controlled release of the active agent from the coating and having release characteristics that are distinct from the first layer.

14. The coating of claim 13, the active agent comprising a hydrophilic active agent.

15. The coating of claim 13, wherein the active agent has a molecular weight of less than 5 kilodaltons and has a water solubility of greater than 10 mg/mL at 25 degrees Celsius.

16. The coating of claim 13, the first layer comprising a polyalkyl(meth)acrylate.

17. The coating of claim 13, the first polymeric layer comprising poly(n-butyl methacrylate) and poly(ethylene-co-vinyl acetate).

18. The coating of claim 13, the second layer comprising a plasma or vapor deposited polymer.

19. The coating of claim 13, the second layer comprising at least one of poly 2-chloro-paraxylylene (parylene C), polyparaxylylene (parylene N), poly 2,5-dichloro-paraxylylene (parylene D).

20. The coating of claim 13, wherein the second layer is selected to produce controlled release comprising a reverse-burst elution profile.

21. A method for producing an article that provides controlled release of a hydrophilic active agent comprising:
depositing a base composition onto a substrate, the composition comprising a hydrophilic active agent,
depositing a protective layer on the base composition, and
depositing an elution control layer on the protective layer, the elution control layer having release characteristics that are distinct from the protective layer.

22. The method of claim 21, wherein the hydrophilic active agent has a molecular weight of less than 5 kilodaltons and has a water solubility of greater than 10 mg/mL at 25 degrees Celsius.

23. The method of claim 21, wherein the elution control layer is selected to produce controlled release comprising a reverse-burst elution profile.
